(19) United States
(12) Patent Application Publication
(10) Pub. No.: US 2022/0134136 A1
(43) Pub. Date: May 5, 2022

(54) ARTHRITIS TREATMENT APPARATUS USING ULTRASONIC WAVE AND FAR-INFRARED RAY

(71) Applicant: Day JOH, Daegu (KR)

(72) Inventor: Day JOH, Daegu (KR)

(21) Appl. No.: 17/123,952

(22) Filed: Dec. 16, 2020

(30) Foreign Application Priority Data
Oct. 29, 2020 (KR) 10-2020-0142258

(51) Int. Cl. A61N 7/02

(52) U.S. Cl. CPC A61N 7/02; A61N 2007/0004

(57) ABSTRACT

An arthritis treatment apparatus including: a treatment part coming into close contact with a patient's skin to transmit far-infrared rays and ultrasonic waves to a deep portion of a joint; and a body for adjusting an internal pressure and ultrasonic wave outputs of the treatment part, wherein the treatment part includes: a heat generating element for transmitting the far-infrared rays to the deep portion of the joint; ultrasonic wave generating elements for transmitting the ultrasonic waves to the deep portion of the joint; and a vacuum chamber configured to locate the heat generating element and the ultrasonic wave generating elements at an interior thereof and to allow the heat generating element and the ultrasonic wave generating elements to come into close contact with the patient's skin.

### CROSS REFERENCE TO RELATED APPLICATION OF THE INVENTION

The present application claims the benefit of Korean Patent Application No. 10-2020-0142258 filed on Oct. 29, 2020, the disclosure of which is incorporated herein by reference in its entirety.

### BACKGROUND OF THE INVENTION

### Field of the Invention

The present invention relates to an arthritis treatment apparatus, and more particularly, to an arthritis treatment apparatus using ultrasonic waves and far-infrared rays.

### Background of the Related Art

Ultrasound therapy is a treatment using ultrasonic waves in the range not audible to humans to obtain joint contracture and adhesion prevention, joint stiffness reduction, pain relief, inflammation recovery, sterilizing effect, fracture healing, and so on.

The ultrasonic waves are sound pressures with frequencies higher than the upper audible limit of human hearing.

Generally, the ultrasonic waves are used in many different fields so as to pass a medium therethrough, to measure reflection signature, or to supply concentrated energy.

A conventional ultrasonic device for treating arthritis is disclosed in Korean Patent No. 10-537343 (Dated on Dec. 12, 2005), and the conventional ultrasonic device includes an ultrasonic wave generator and an ultrasonic transducer.

Because of piezoelectric elements having coin-like round shapes generally used, however, the conventional ultrasonic device fails to allow ultrasonic waves to penetrate deep portions within tissue cells, and because of curved skin, besides, the conventional ultrasonic device does not come into close contact with a patient's skin at a high density, thereby failing to allow the ultrasonic waves to penetrate the skin efficiently.

Accordingly, there is a need for developing a technology in which the skin activated though the transmission of ultrasonic waves and far-infrared rays comes into close contact with a treatment device by means of vacuum suction to thus induce cellular respiration of skin, so that the pain of articular cells can be effectively relieved and the regeneration effect of the chondrocytes can be optimized.

### SUMMARY OF THE INVENTION

Accordingly, the present invention has been made in view of the above-mentioned problems occurring in the related art, and it is an object of the present invention to provide an arthritis treatment apparatus that is capable of coming into close contact with the skin activated though the transmission of ultrasonic waves and far-infrared rays by means of vacuum suction to thus induce cellular respiration of skin, so that the pain of articular cells can be effectively relieved and the regeneration effect of the chondrocytes can be optimized.

To accomplish the above-mentioned objects, according to the present invention, there is provided an arthritis treatment apparatus including: a treatment part coming into close contact with a patient's skin to transmit far-infrared rays and ultrasonic waves to a deep portion of a joint; and a body for adjusting an internal pressure and ultrasonic wave outputs of the treatment part, wherein the treatment part includes: a heat generating element for transmitting the far-infrared rays to the deep portion of the joint; ultrasonic wave generating elements for transmitting the ultrasonic waves to the deep portion of the joint; and a vacuum chamber configured to locate the heat generating element and the ultrasonic wave generating elements at an interior thereof and to allow the heat generating element and the ultrasonic wave generating elements to come into close contact with the patient's skin.

According to the present invention, desirably, the treatment part further includes a housing configured to locate the ultrasonic wave generating elements at an interior thereof and to allow the heat generating element to be attached to one surface thereof.

According to the present invention, desirably, the treatment part further includes a temperature sensor for measuring a temperature of the skin, and the body includes a controller for adjusting output periods of the ultrasonic wave generating elements if the measured temperature of the skin is greater than a reference value set in advance.

According to the present invention, desirably, the body further includes a vacuum pump connected to the vacuum chamber by means of a vacuum tube to adjust the internal pressure of the vacuum chamber.

According to the present invention, desirably, the treatment part further includes a valve connected between the vacuum chamber and the vacuum tube, and the valve is blocked under the control of the controller if a pressure generated from the vacuum pump is greater than a reference value set in advance.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1 is a block diagram showing an arthritis treatment apparatus according to the present invention;

FIGS. 2 and 3 are views showing a treatment part of the arthritis treatment apparatus according to the present invention;

FIG. 4 is a perspective view showing an ultrasonic wave generating element of the arthritis treatment apparatus according to the present invention;

FIG. 5 is a perspective view showing a housing of the arthritis treatment apparatus according to the present invention; and

FIG. 6 is a perspective view showing another example of the ultrasonic wave generating element of the arthritis treatment apparatus according to the present invention.

### DETAILED DESCRIPTION OF THE EMBODIMENTS

The present invention is disclosed with reference to the attached drawings wherein the corresponding parts in the embodiments of the present invention are indicated by corresponding reference numerals and the repeated explanation on the corresponding parts will be avoided. If it is determined that the detailed explanation on the well known technology related to the present invention makes the scope of the present invention not clear, the explanation will be avoided for the brevity of the description.

In the description, when it is said that one portion is described as “includes” any component, one element further may include other components unless no specific description is suggested.

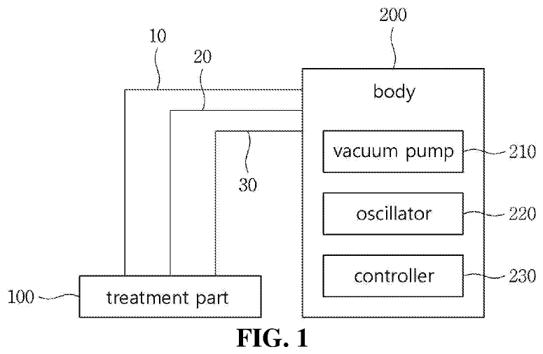

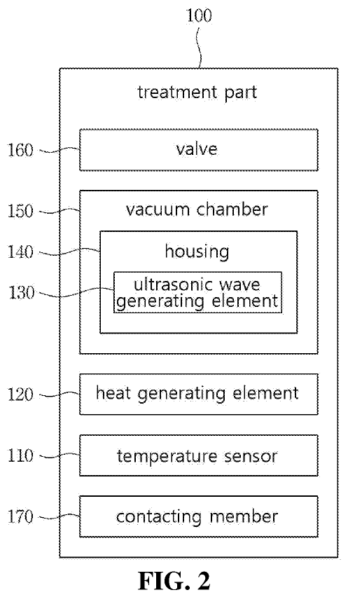

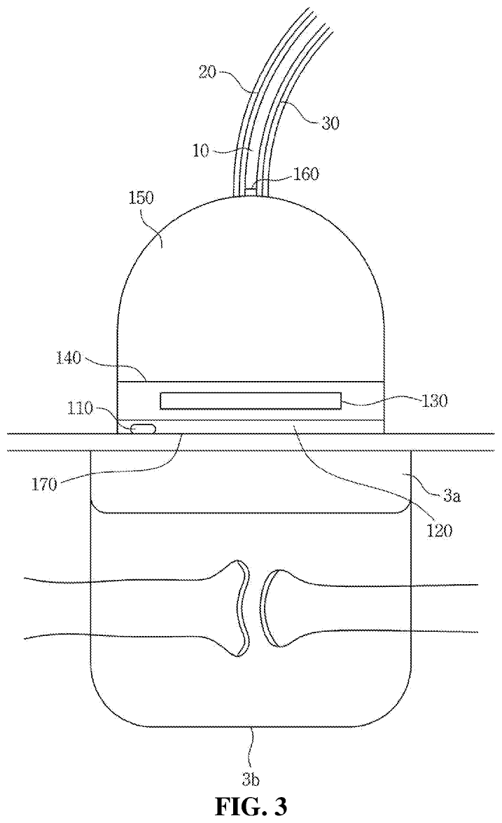

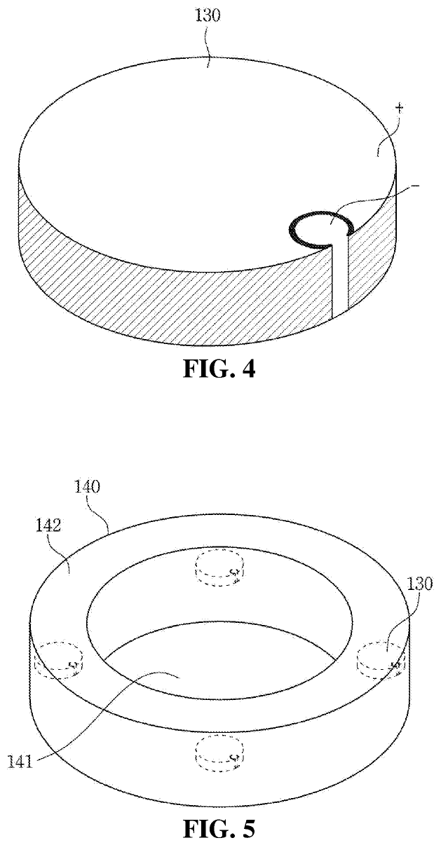

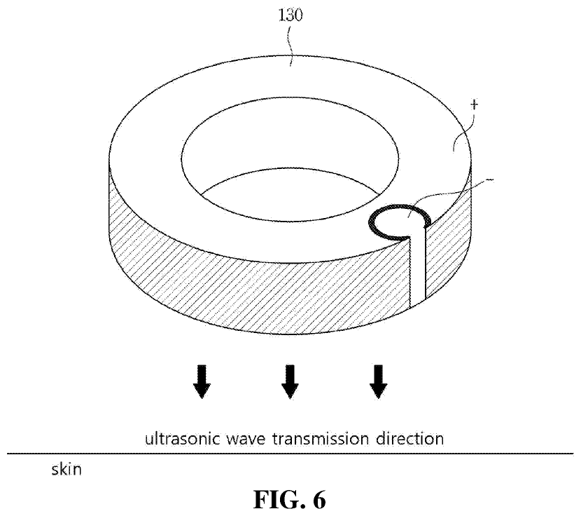

FIG. 1 is a block diagram showing an arthritis treatment apparatus according to the present invention, FIGS. 2 and 3 are views showing a treatment part of the arthritis treatment apparatus according to the present invention, FIG. 4 is a perspective view showing an ultrasonic wave generating element of the arthritis treatment apparatus according to the present invention, FIG. 5 is a perspective view showing a housing of the arthritis treatment apparatus according to the present invention, and FIG. 6 is a perspective view showing another example of the ultrasonic wave generating element of the arthritis treatment apparatus according to the present invention.

Referring to FIG. 1, an arthritis treatment apparatus according to the present invention includes a treatment part 100 and a body 200.

The treatment part 100 comes into close contact with a patient's skin to transmit far-infrared rays and ultrasonic waves to a deep portion of a joint.

Referring to FIGS. 2 and 3, the treatment part 100 according to the present invention includes a temperature sensor 110, a heat generating element 120, ultrasonic wave generating elements 130, a housing 140, a vacuum chamber 150, a valve 160, and a contacting member 170.

According to the present invention, the temperature sensor 110 is located inside the vacuum chamber 150 to measure a skin temperature of the patient.

The temperature sensor 110 transmits the measured skin temperature information to a controller 230 as will be discussed later through a temperature sensing line 20.

According to the present invention, the heat generating element 120 is located inside the vacuum chamber 150 to transmit the far-infrared rays to the deep portion of the joint. Through the heat generating element 120, the far-infrared rays can be transmitted to a portion 3a located at a depth of about 1 to 2 cm on the skin surface.

According to the present invention, the heat generating element 120 is a far-infrared ray generating element having a housing made of a metal or ceramic material.

The heat generating element 120 receives an output control signal from the controller 230 through an ultrasonic wave signal line 30.

According to the present invention, the ultrasonic wave generating elements 130 are located inside the vacuum chamber 150 to transmit ultrasonic waves to the deep portion of the joint. Through the ultrasonic wave generating elements 130, the ultrasonic waves can be transmitted to a portion 3b located at a depth of about 2 to 10 cm on the skin surface.

According to the present invention, the ultrasonic wave generating elements 130 are piezoelectric elements.

Referring to FIG. 4, a positive pole + and a negative pole − of each ultrasonic wave generating element 130 are coated with a silver (Ag) material. The silver coated on the negative pole − is connected up to the top of the piezoelectric element, and in transmitting the ultrasonic waves and heat to a medium, a beating phenomenon (vortex/helix turn) occurs. The beating phenomenon is an interference phenomenon occurring in the same frequencies or when two or more frequencies are superposed, so that the harmony of waves is broken to cause a spiral and circular motion like a vortex. As a result, blood circulation of inflamed or hard cellular tissues can be gentle.

Through the heat generating element 120 and the ultrasonic wave generating elements 130, the ultrasonic waves and the far-infrared rays can be simultaneously transmitted to the joint (e.g., the patella), so that through deep heat transmission and inflammation removal, the capillary vessels can be activated. In this case, the skin activated though the transmission of the ultrasonic waves and the far-infrared rays comes into close contact with the treatment part 100 by means of vacuum suction to thus induce the cellular respiration of the skin, thereby effectively releasing the pain of the articular cells and optimizing the regeneration effect of the chondrocytes.

Through the ultrasonic cavitation, also, the waves and heat are transmitted to the deep portion of the joint, and through the vacuum suction, gas exchange starting at the capillary vessels of the skin is optimized, thereby effectively removing the inflammation of the joint.

Referring to FIG. 5, an example of the housing 140 is provided, and in this case, the housing 140 is configured to have a hole 141 formed on the center thereof, to locate the ultrasonic wave generating elements 130 at an interior 142 thereof, and to attach the heat generating element 120 to one surface (toward the skin) thereof. Through the hole 141 formed on the center of the housing 140, the vacuum suction can occur.

The housing 140 may have the shapes of a torus, a circle, a triangle, and a square. The housing 140 serves to surround the ultrasonic wave generating elements 130 and also serves as a lens. Through the housing 140 having the shape of the torus, the heat and waves can be effectively transmitted to the skin.

The heat generating element 120 is attached to one surface of the housing 140.

Referring to FIG. 6, the ultrasonic wave generating element 130 has the shape of a torus. Top and underside of the ultrasonic wave generating element 130 are coated with a silver (Ag) material.

According to the present invention, a positive pole + and a negative pole − are formed on top of each ultrasonic wave generating element 130, so that a beating phenomenon (which means that heat accumulated in heat storage reaction rotates in a clockwise or counterclockwise direction) occurs in the heat and waves transmitted to the hard cellular tissues of the deep portion at the inside of the skin to which the ultrasonic waves have been transmitted. As the positive pole + and the negative pole − are formed on top of each ultrasonic wave generating element 130, the inflammation or reactive oxygen species in the tissue cells is decreased, and activities of blood vessels become gentle.

According to the present invention, the vacuum chamber 150 is configured to locate the heat generating element 120 and the ultrasonic wave generating elements 130 at the inside thereof and to allow the heat generating element 120 and the ultrasonic wave generating elements 130 to come into close contact with the skin through the suction of vacuum pulses. As a result, a density of skin becomes high and the transmission efficiencies of the ultrasonic waves and the far-infrared rays become improved. Through the suction of the vacuum pulses, further, the joint inflammation and gas can be discharged to the outside of the skin.

According to the present invention, the valve 160 is a vacuum backflow prevention valve connected to a connected portion between a vacuum tube 10 and the vacuum chamber 150. According to the present invention, the valve 160 is a solenoid valve.

According to the present invention, the contacting member 170 is a rubber band to which the vacuum suction of the ultrasonic wave generating elements 130 is applied. According to the present invention, the contacting member 170 has a solid shape three-dimensionally printed according to the patient's joint portion. As a result, the contacting member 170 can come into close contact with the patient's skin airtightly, regardless of the curve of the skin.

According to the present invention, the body 200 includes a vacuum pump 210, an oscillator 220, and the controller 230.

The vacuum pump 210 is connected to the vacuum chamber 150 by means of the vacuum tube 10 and adjusts an internal pressure of the vacuum chamber 150 through suction.

The oscillator 220 adjusts the ultrasonic wave outputs from the ultrasonic wave generating elements 130.

According to the present invention, if a temperature of the skin measured through the temperature sensor 110 is greater than a reference value (for example, 43°) set in advance, the controller 230 controls the oscillator 220 to adjust output periods of the ultrasonic wave generating elements 130. For example, if a temperature of the skin measured through the temperature sensor 110 is 43°, the controller 230 controls the oscillator 220 to change the outputs of the ultrasonic wave generating elements 130 from a continuous mode to an intermittent mode.

According to the present invention, if a pressure generated from the vacuum pump 210 is greater than a reference value (for example, 500 mHg, 550 mHg, or 600 mHg) set in advance, the valve 160 is blocked under the control of the controller 230.

The body 200 is operated with the power received from the outside.

As described above, the arthritis treatment apparatus according to the present invention can transmit the ultrasonic waves and the far-infrared rays simultaneously to the deep portion of the joint through the heat generating element and the ultrasonic wave generating elements, so that through deep heat transmission and inflammation removal, the capillary vessels can be activated. In this case, the skin activated through the transmission of the ultrasonic waves and the far-infrared rays comes into close contact with the treatment part by means of the vacuum suction to thus induce the cellular respiration of skin, thereby effectively releasing the pain of the articular cells and optimizing the regeneration effect of the chondrocytes.

Through the ultrasonic cavitation, also, the waves and heat are transmitted to the deep portion of the joint, and through the vacuum suction, gas exchange starting at the capillary vessels of the skin is optimized, thereby effectively removing the inflammation of the joint.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

What is claimed is:

1. An arthritis treatment apparatus comprising:
a treatment part coming into close contact with a patient's skin to transmit far-infrared rays and ultrasonic waves to a deep portion of a joint; and
a body for adjusting an internal pressure and ultrasonic wave outputs of the treatment part,
wherein the treatment part comprises:
a heat generating element for transmitting the far-infrared rays to the deep portion of the joint;
ultrasonic wave generating elements for transmitting the ultrasonic waves to the deep portion of the joint; and
a vacuum chamber configured to locate the heat generating element and the ultrasonic wave generating elements at an interior thereof and to allow the heat generating element and the ultrasonic wave generating elements to come into close contact with the patient's skin.

2. The arthritis treatment apparatus according to claim 1, wherein the treatment part further comprises a housing configured to locate the ultrasonic wave generating elements at an interior thereof and to allow the heat generating element to be attached to one surface thereof.

3. The arthritis treatment apparatus according to claim 1, wherein the treatment part further comprises a temperature sensor for measuring a temperature of the skin, and the body comprises a controller for adjusting output periods of the ultrasonic wave generating elements if the measured temperature of the skin is greater than a reference value set in advance.

4. The arthritis treatment apparatus according to claim 3, wherein the body further comprises a vacuum pump connected to the vacuum chamber by means of a vacuum tube to adjust the internal pressure of the vacuum chamber.

5. The arthritis treatment apparatus according to claim 4, wherein the treatment part further comprises a valve connected between the vacuum chamber and the vacuum tube, and the valve is blocked under the control of the controller if a pressure generated from the vacuum pump is greater than a reference value set in advance.
